(19) United States
(12) Patent Application Publication
(10) Pub. No.: US 2007/0116788 A1
(43) Pub. Date: May 24, 2007

(54) METHOD OF ENHANCING MOTOR FUNCTION

(75) Inventors: Takatoshi MURASE, Haga-gun (JP); Satoshi Haramizu, Haga-gun (JP); Noriyasu Ota, Haga-gun (JP); Misako Inaba, Haga-gun (JP); Akira Shimotoyodome, Haga-gun (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: KAO CORPORATION, Tokyo (JP)

(21) Appl. No.: 11/626,032

(22) Filed: Jan. 23, 2007

Related U.S. Application Data
(63) Continuation of application No. 11/045,312, filed on Jan. 31, 2005.

(51) Int. Cl. A61K 31/353; A61K 36/82

(52) U.S. Cl. 424/729; 514/456

(57) ABSTRACT

This invention relates to a method for enhancing physical activities by administering catechins. The combined use of catechin ingestion and exercise makes it possible to promote the oxidation of fat and hence, to inhibit obesity, liver enlargement and diabetes.

### TECHNICAL FIELD

This invention relates to a method for enhancing physical activities in a broader sense including exercise and labor.

### BACKGROUND ART

Improvements in physical activities are considered to be important not only in competitive sports but also from the viewpoint of prevention and amelioration of obesity and life-style related diseases. Regular exercise is recommended for the prevention and amelioration of obesity and life-style related diseases, and its importance is widely acknowledged. Nonetheless, it is not easy to actually do regular exercise in everyday life. If it becomes possible to enhance the effects of exercise performed in our daily life, the effect of such limited exercise is expected to be further heightened and hence, to contribute to the prevention and amelioration of obesity and life-style related diseases.

However, little is known about ingredients capable of enhancing the effects of exercise insofar as we can acknowledge.

In the meantime, catechins contained in green tea, grapes, cacao beans and the like have been reported to bring physiological benefits such as suppressing effect on an increase in cholesterol level (Patent Document 6), inhibitory effect on α-amylase activity (Patent Document 7), inhibitory effect on an elevation of blood sugar level (Patent Document 8), arteriosclerosis-preventing effect (Patent Document 9), anti-oxidation effect (Patent Document 10), antimicrobial effect (Patent Document 11), suppressing effect on an increase in blood pressure and inhibitory effect on enzymatic activities (Patent Document 12), antiulcer effect (Patent Document 13), and mutation inhibitory effect. However, absolutely nothing is known as to how catechins influence the physical functions.
[Patent Document 1] JP-A-60-156614
[Patent Document 2] JP-A-03-133928
[Patent Document 3] JP-A-04-253918
[Patent Document 4] JP-A-04-352726
[Patent Document 5] JP-B-01-044234
[Patent Document 6] JP-A-02-276562
[Patent Document 7] JP-A-03-133928
[Patent Document 8] JP-A-63-277628

### DISCLOSURE OF THE INVENTION

The present invention provides a method for enhancing physical functions and also to a method for inhibiting obesity, diabetes, liver enlargement or the like, each of which contains administering an effective amount of catechins.

### BRIEF DESCRIPTION OF THE DRAWINGS

FIG. 1 Diagrams showing expiration analysis results when the ingestion of catechins and exercise were used in combination.

FIG. 2 Diagram showing the results on fat oxidation when the ingestion of catechins and exercise were used in combination.

### MODES FOR CARRYING OUT THE INVENTION

This invention provides a physical activity enhancing method effective for the inhibition of obesity.

The present inventors have proceeded with an investigation on physiological effects brought about by catechins, and have unexpectedly found that catechins have an excellent effect for the enhancement of physical functions.

According to the present invention, physical functions are enhanced and the effectiveness of exercise is heightened, thereby effectively bringing about inhibitory effects for obesity, diabetes and liver enlargement.

In general, the term “catechins” is a generic term, which encompasses catechin, gallocatechin, catechingallate, gallocatechingallate, epicatechin, epigallocatechin, epicatechingallate and epigallocatechingallate. In the present invention, catechins may contain one or more of these compounds. Further, plant extracts containing catechins, such as tea extracts can be used.

Catechins, an ingredient for use in the present invention, can be extracted with water or hot water from tea leaves prepared from green tea leaves obtainable from the Genus Camellia, for example, C. sinensis, C. assamica or the Yabukita variety, or a hybrid thereof, and in some instances, the extraction can be conducted with an extraction aid added to water or hot water. Such prepared tea leaves include (1) green teas such as sencha (middle-grade green tea), bancha (coarse green tea), gyokuro (shaded green tea), tencha (powdered tea) and kamairicha (roasted tea); (2) semi-fermented teas such as tekkannon (Teguajin), irotane, ougonkei (huang jin gui) and buigancha (Wuyiyancha), all of which are collectively called “oolong tea”; and (3) fermented teas called “black tea”, such as Darjeeling, Ceylon Uva and Chinese Keemun. As an extraction method of tea, the extraction can be effected by a conventional method such as stirring extraction. Upon extraction, an organic acid or an organic acid salt such as sodium ascorbate may be added to water. It is also possible to make combined use of boiling deaeration or an extraction method which is conducted while bubbling an inert gas such as nitrogen gas to eliminate dissolved oxygen, that is, under a so-called non-oxidizing atmosphere. Instead of directly extracting from tea leaves, it is also possible to add a concentrate or purified product of a tea extract.

The term “the concentrate of a tea extract” as used herein means one obtained by concentrating an extract of tea leaves in hot water or a water-soluble organic solvent, while the term “the purified product of a tea extract” as used herein means one obtained by conducting purification with a solvent and a column. Examples include those prepared by the processes exemplified in detail in JP-A-59-219384, JP-A-04-0200589, JP-A-05-260907, JP-A-05-306279 and the like. Commercially-available products include “POLYPHENON” (product of Tokyo Food Techno Co., Ltd.), “TEAFURAN” (product of ITO EN, LTD.), “SUNPHENON” (product of Taiyo Kagaku Co., Ltd.), “SUN OOLONG” (product of Suntory Limited), etc. As catechins, on the other hand, it is possible to use products obtained from other raw material sources, for example, grapes and products obtained by processing grapes such as wine, juice or the like, cacao beans and those obtained by processing cacao beans as a raw material, and even chemically synthesized products. As the forms of a concentrate of a tea extract and a purified product of a tea extract, various forms can be mentioned such as solids, aqueous solutions and slurries. As a liquid for dissolving or diluting the concentrate of the tea extract or the purified product of the tea extract, water, carbonated water, a conventional tea extract or the like can be mentioned.

As catechins in the present invention, a concentrate of a tea extract or a purified product of a tea extract is generally used from the view point of availability and cost. In particular, the use of a concentrate of a green tea extract or a purified product of a green tea extract is preferred.

The administration of catechins in combination with, for example, regular exercise, as described subsequently in examples, promotes energy expenditure and fat oxidation not only during exercise but also when one is not exercising, and further, enhances the anti-obesity effect and anti-diabetes effect of exercise so that physical activities are enhanced.

In the present invention, catechins can be administered to human and animals, and can also be ingested by adding them to various beverages, medicines, pet foods and the like. Catechins may also be ingested in the form of foods or beverages. The ingestion of catechins can enhance physical functions and, when combined with regular exercise, can enhance fat oxidation promoting effect, anti-obesity effect and the like. As foods, catechins can be applied to beverages and foods, invalid diets and specific health foods, all of which make use of the physiological functions of catechins. When employed as medicines, catechins can be formulated into oral solid preparations such as tablets and granules and also into oral liquid preparations such as liquid medicines and syrups.

To formulate an oral solid preparation, tablets, coated tablets, granules, a powder, capsules or the like can be produced by a method known per se in the art followed by the addition of an excipient and optionally, a binder, disintegrator, lubricant, color, taste corrigent, aroma corrigent and the like are added to catechins. To formulate an oral liquid preparation, on the other hand, a liquid medicine, syrup, elixir or the like can be produced by a method known per se in the art followed by the addition of a taste corrigent, buffer, stabilizer, taste corrigent and the like are added to catechins.

The content of catechins in each of the above-described preparations varies depending upon the manner of its use, and in the case of beverages, foods, pet foods or the like, can be generally from 0.01 to 5 wt %, preferably from 0.05 to 5 wt %, more preferably from 0.1 to 1 wt %. In the case of products other than those described above, for example, medicines including oral solid preparations such as tablets, granules and capsules and oral liquid preparations such as liquid medicines and syrups, the content of catechins can be generally from 0.01 to 95 wt %, preferably from 5 to 95 wt %, more preferably from 10 to 95 wt %.

The effective ingestion rate of catechins may be preferably from 100 to 3,000 mg/60 kg body-weight, more preferably at from 250 to 2,000 mg/60 kg body-weight, even more preferably at from 250 to 1,000 mg/60 kg body-weight, per day.

### EXAMPLES

The present invention will next be described in detail based on examples. It should, however, be borne in mind that the present invention is by no means limited to these examples.

### Example 1

After C57BL/6J mice (male, 6 weeks old) were acclimatized for 1 week to treadmill running (25 m/min, 30 minutes/day), they were divided into two groups, that is, an exercise group (Group 1) and an exercise+tea catechin ingestion group (Group 2), (N=10/group) such that the body weights became equal. Subsequent to the division into the groups, the mice were allowed to ingest for 4 weeks the corresponding test feed described in Table 1. Both groups were given exercise on a treadmill (25 m/min, 30 minutes, twice/week), and further, an expiration analysis was conducted once a week during exercise. As a rearing environment, the room temperature, humidity and lighting time were set at 23±2° C., 55±10%, and from 7:00 to 19:00. On the last day of the test, each mouse was dissected under anesthesia shortly after exercise, and the liver and muscle (the gastrocnemius) were collected and used for the measurement of lipid metabolism activating effect (β oxidation activity).

TABLE 1

| (%) | Group 1 | Group 2 |
|---|---|---|
| Casein | 20 | 20 |
| Triglycerides | 25 | 25 |
| Lard | 5 | 5 |
| Tea catechins | 0 | 0.5 |
| Sucrose | 13 | 13 |
| Minerals | 3.5 | 3.5 |
| Vitamins | 1 | 1 |
| Cellulose | 4 | 4 |
| Potato starch | 28.5 | 28 |
| Exercise | Given | Given |

<Expiration Analysis>

Oxygen expenditure (VO₂) and carbon dioxide excretion (VCO₂) were measured at intervals of 5 minutes by “OXYMAX SYSTEM Version 5.61” (manufactured by Columbus Instruments) During the measurement, the running speed was controlled in accordance with the following program:

10 m/min, 5 minutes
→ 15 m/min, 10 minutes
   → 10 m/min, 30 minutes.

The flow rate of air through a chamber was set at 0.3 L/min, and the sampling flow rate of air from the chamber was set at 0.8 L/min. The calorific value was calculated in accordance with the following formula:
Calorific value (kcal/min)=CV×VO₂(mL/min)×0.001CV(cal/mL)=3.815+1.232×RQ²¹ Fat oxidation (mg/g−body weight/min)=1.67×(1−RQ)×VO₂/1000
CV: calorific value calculated based on respiratory exchange ratio
RQ: respiratory quotient RQ=carbon dioxide excretion/oxygen expenditure
   <Measurement of Lipid Metabolism Activating Effect>

Each collected liver or muscle (the gastrocnemius) was homogenized in a buffer (250 mM sucrose, 1 mM EDTA in 10 mL HEPES (pH 7.2), and insoluble tissue residues were removed by centrifugation and a supernatant was obtained, the protein content of which was measured subsequently. Individual samples were adjusted to have the same protein concentration, and were provided for the measurement of lipid metabolism activating effect (β poxidationactivity). In a buffer of 200-μL final volume (50 mM Tris-HCl (pH 8.0), 40 mM NaCl, 2 mM KCl, 2 mM MgCl₂, 1 mM DTT, 5 mM ATP, 0.2 mM L-carnitine, 0.2 mM NAD, 0.06 mM FAD, 0.12 mM CoA, 3 mM α-cyclodextrin), the supernatant protein (100 μg) was reacted with [¹⁴C]-palmitic acid (0.1 μCi) at 37° C. for 20 minutes. After the reaction was terminated with 0.6 N perchloric acid (200 μL), unreacted [ ⁴C]-palmitic acid was eliminated three times with hexane (1 mL, each). The radioactivity of the water layer was measured to determine the lipolysis activity.

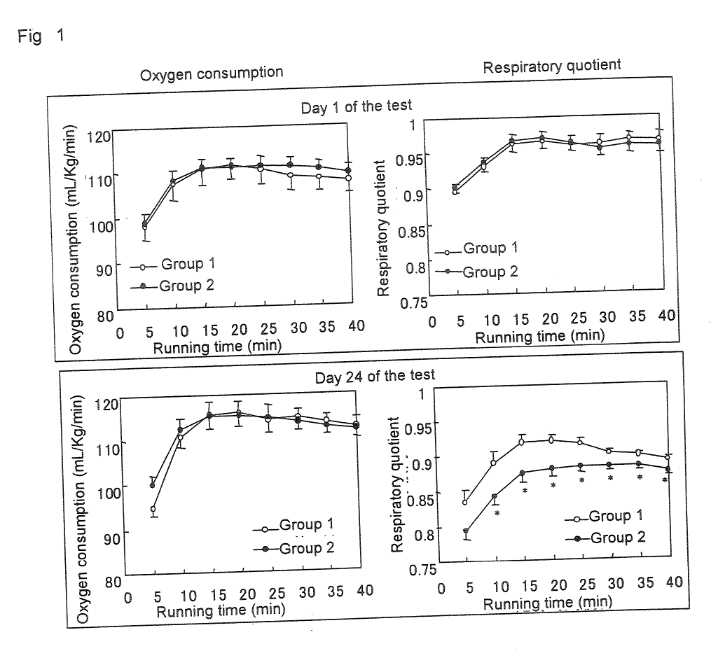

The results of an expiration analysis are shown in FIG. 1. On Day 1 of the test, Group 1 and Group 2 had substantially the same value. On Day 24 after the initiation of the test, a significant reduction in respiratory quotient was observed in Group 2 compared with Group 1. This reduction in respiratory quotient under the conditions of combined use of catechins and exercise means an increase in the metabolization rate of lipid energy.

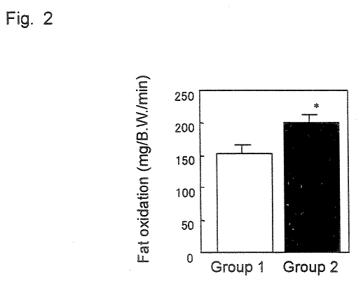

The results of fat oxidation on Day 24 after the initiation of the test are shown in FIG. 2. On Day 1 after the initiation of the test, no significant difference was observed in fat oxidation between both groups. On Day 24 after the initiation of the test, however, the fat oxidation increased significantly in Group 2 compared with Group 1. From the foregoing, it has become clear that catechins have an effect to promote the oxidation of fat under the conditions of combined use with exercise.

The measurement results of lipid metabolism (β oxidation activity) activity are shown in Table 2. Assuming that the lipolysis activity of Group 1 was 100, the lipolysis activity of Group 2 was indicated in terms of a relative value. By ingesting catechins while doing exercise, the hepatic and muscular, lipid metabolism activities increased markedly. As it has been confirmed that the muscular lipid metabolism activity does not increase even when catechins are ingested under the conditions of no exercise, catechins are considered to exhibit their effect for the first time when they are ingested in combination with exercise. In other words, catechins by themselves are not equipped with muscular liquid metabolism activating effect but, when combined with exercise, enhance physical activities so that effects of exercise can be heightened. As a result, catechins are considered to show excellent lipid metabolism promoting effect.

TABLE 2

| | Group 1 | Group 2 |
|---|---|---|
| Muscular lipid metabolism activity | 100 | 158* |
| Hepatic lipid metabolism activity | 100 | 155* |

*P < 0.05

### Example 2

C57BL/6J mice (male, 6 weeks old) were used in the experiment. After those mice were provisionally reared for 1 week, they were divided into 5 groups, that is, a low-fat feed group (Group 1), a high-fat feed group (Group 2), a high-fat feed+tea catechin ingestion group (Group 3), a high-fat feed+exercise group (Group 4) and a high-fat feed+catechins+exercise group (Group 5) such that the body weights became equal.

After having been divided into five groups, the mice were allowed to ingest for 15 weeks the corresponding test feed shown in Table 3. Exercise was given to Group 4 and Group 5 in a running water pool (6 L/min, 30 minutes, 3 times/week) during the test period. Throughout the test period, the high-fat feed ingestion groups were fed such that the ingestion rate became equal from one cage to another. As a rearing environment, the room temperature, humidity and lighting time were set at 23±2° C., 55±10%, and from 7:00 to 19:00.

TABLE 3

| (%) | Group 1 | Group 2 | Group 3 | Group 4 | Group 6 |
|---|---|---|---|---|---|
| Casein | 20 | 20 | 20 | 20 | 20 |
| Triglycerides | 5 | 20 | 20 | 20 | 20 |
| Lard | 0 | 10 | 10 | 10 | 10 |
| Tea catechins | 0 | 0 | 0.5 | 0 | 0.5 |
| Sucrose | 0 | 13 | 13 | 13 | 13 |
| Minerals | 3.5 | 3.5 | 3.5 | 3.5 | 3.5 |
| Vitamins | 1 | 1 | 1 | 1 | 1 |
| Cellulose | 4 | 4 | 4 | 4 | 4 |
| Potato starch | 66.5 | 28.5 | 28 | 28.5 | 28 |
| Exercise | Not given | Not given | Not given | Given | Given |

The running water pool had dimensions of 90×45×45 cm (length×width×depth) and was made of acrylic resin. Water was filled to a depth of 38 cm, and was controlled with an electric heater such that the water temperature remained constant at 34° C. Further, the flow rate was controlled to be constant by an electric pump (“TYPE C-P60H”, Hitachi, Tokyo, Japan). The flow rate was measured by a digital flow rate meter (“MODEL SV-101-25S”; Sankou, Tokyo, Japan).

After the test, each mouse was exsanguinated from the abdominal vena cava under anesthesia with sevofrane, was sacrificed, and then, dissected. The liver and fatty tissues (epididymis-surrounding fat, kidney-surrounding fat, and retroperitoneal fat) were collected, and the weights of the respective tissues were measured. The muscle and liver tissues were frozen with liquefied nitrogen immediately after the collection, and were stored at −80° C. until their use in experiments. Using a tube with an anticoagulant (EDTA-2Na) contained therein, the blood was centrifuged (10,000 rpm, 10 min, 4° C.) after its collection, and then, serum was obtained.

The body weights, liver weights, and body fat weights (total values of epididymis-surrounding fat, kidney-surrounding fat, and retroperitoneal fat) at the time of dissection are shown in Table 4.

TABLE 4

| | Group 1 | Group 2 | Group 3 | Group 4 | Group 5 |
|---|---|---|---|---|---|
| Body | 32.3 a | 41 b | 37.8 c | 38.4 cd | 35.1 d |
| weight | | | | | |
| (g) | | | | | |
| Liver (g) | 1.21 a | 1.52 b | 1.36 c | 1.32 c | 1.17 a |
| Body fat | 1.06 a | 2.86 b | 2.31 c | 2.3 c | 1.85 d |
| weight | | | | | |
| (g) | | | | | |

The different alphabets indicate the recognition of significant differences among the groups (P < 0.05).

As shown in Table 4, compared with the low-fat feed ingestion group (Group 1), the body weight increased with significance in the high-fat feed ingestion group (Group 2) so that the mice gained weight dependent upon the high-fat feed. In the catechin group (Group 3) and exercise group (Group 4), on the other hand, the body weight decreased compared with Group 2. Accordingly, the ingestion of catechins and exercise are considered to be effective for the inhibition of obesity. In the catechins+exercise group (Group 5), the body weight decreased more pronouncedly compared with Groups 2, 3 and 4. It has, therefore, become evident that catechins enhance the effects of exercise and are effective for more effectively inhibiting obesity. Further, the body fat weight also increased markedly in Group 1 compared with Group 2. The accumulation of such body weight was inhibited by catechins (Group 3) or exercise (Group 4), and was pronouncedly inhibited by the combination of exercise and catechins (Group 5). It has, therefore, become evident that, when combined with exercise, catechins enhance the effect of exercise for inhibiting the accumulation of body fat and inhibit obesity more effectively.

Compared with Group 1, the liver weight increased in Group 2 so that liver enlargement was confirmed. In Group 3 and Group 4, lower values were indicated compared with Group 2, and in Group 5, a still lower value was indicated. It has, therefore, become evident that the combined use of catechins and exercise can effectively inhibit liver enlargement.

The results of a blood analysis are shown in Table 5.

TABLE 5

| | Group 1 | Group 2 | Group 3 | Group 4 | Group 5 |
|---|---|---|---|---|---|
| Glucose | 214 a | 263 b | 229 ab | 230 ab | 210 b |
| (mg/dL) | | | | | |
| Insulin | 1.23 a | 6.47 b | 5.18 bc | 4.48 c | 2.31 ad |
| (ng/mL) | | | | | |
| Leptin | 8.89 a | 39.56 b | 22.9 c | 20.37 c | 13.32 ad |
| (ng/mL) | | | | | |

The different alphabets indicate the recognition of significant differences among the groups (P < 0.05).

Compared with Group 1, the blood sugar (glucose) level increased in Group 2. In Group 3 and Group 4, on the other hand, lower values were indicated compared with Group 2, and in Group 5, a still lower value was indicated. It has, therefore, become evident that the combined use of catechins and exercise can effectively inhibit an elevation in blood sugar level. Catechins are, therefore, considered to more effectively inhibit diabetes when combined with exercise.

Compared with Group 1, the blood insulin level similarly increased in Group 2. In Group 3 and Group 4, on the other hand, lower values were indicated compared with Group 2, and in Group 5, a still lower value was indicated. The combined used of catechins and exercise is, therefore, considered to inhibit the onset of insulin resistance so that the insulin sensitivity is maintained well. Catechins are, therefore, considered to more effectively inhibit diabetes when combined with exercise.

Concerning leptin, the blood leptin level increased in Group 2 compared with Group 1. In Group 3 and Group 4, on the other hand, lower values were indicated compared with Group 2, and in Group 5, a still lower value was indicated. The combined used of catechins and exercise is, therefore, considered to inhibit the onset of leptin resistance so that the leptin sensitivity is maintained well. Catechins are, therefore, considered to more effectively inhibit obesity and diabetes when combined with exercise.

### Example 3

Control beverages (500 mL per bottle) with no tea catechins contained therein and catechin beverages (500 mL per bottle) with the tea catechins described in the table and contained therein were prepared, and were used in the test. Those two kinds of test beverages were prepared such that they contained, as additives, a sweetener (sugar, sugar alcohol or the like), a sour seasoning (citric acid or the like), an electrolyte (Na salt, K salt, or the like), an antioxidant and a flavorant, had a calorific value of 5 kcal/100 mL and a caffeine concentration lower than 8 mg/100 mL, and became identical in the ingredients other than catechins. The composition of the tea catechins used in the test beverages is shown in Table 6.

TABLE 6
Composition of Catechins in Test Beverages

| | | | Catechin |
|---|---|---|---|
| | | Control | beverage |
| Catechin | (mg/500 mL) | 0.0 | 12.7 |
| Epicatechin | (mg/500 mL) | 0.0 | 49.7 |
| Gallocatechin | (mg/500 mL) | 0.0 | 36.0 |
| Epigallocatechin | (mg/500 mL) | 0.0 | 174.7 |
| Catechin gallate | (mg/500 mL) | 0.0 | 4.1 |
| Epicatechin gallate | (mg/500 mL) | 0.0 | 65.7 |
| Gallocatechin gallate | (mg/500 mL) | 0.0 | 9.1 |
| Epigallocatechin gallate | (mg/500 mL) | 0.0 | 218.4 |
| Total | (mg/500 mL) | 0.0 | 570.4 |

Normal male subjects were divided into two groups such as the BMIs (Body Mass Index: body weight (kg)/height (m)²) and the expiration analysis data measured as described below become substantially equal (control group, n=7; catechin group, n=7), and were directed to ingest the corresponding test beverage for 2 months. During the test period, each subject ingested the corresponding test beverage as much as 500 mL per day, and did treadmill exercise at 5 km per hour for 30 minutes three times a week. After the ingestion for 2 months, an expiration analysis was conducted to measure the energy expenditure at rest and also, during treadmill exercise.

During two days before the expiration measurement, each subject was directed to ingest predetermined meals (2,200 kcal, fat: 55 g/day) for breakfast, lunch and supper. During that period, he was temperated and was also directed to refrain from eating between meals. After the supper on the day preceding the measurement, he was prohibited to ingest anything other than predetermined drinking water (fasted for 13 hours).

From 2 hours ahead of his entrance to a measurement room on the day of measurement, he was prohibited to take drinking water, and was also prohibited to do any activities that would be considered to affect the expiration analysis, such as the use of stairs. Using “VO2000 METABOLIC TESTING SSYTEM” (Medical Graphics Corporation; U.S.A.), the energy expenditure was measured in the measurement room, the temperature and humidity of which were controlled at 22° C. and 40%, respectively. After the entrance to the measurement room, the subject was acclimatized to a sitting position at rest for 30 minutes and was directed to ingest the test beverage (500 mL) in about 3 minutes. Subsequent to the ingestion, a resting expiration analysis was conducted for 30 minutes in a sitting position with the eyes closed, followed by an exercising expiration analysis while walking at 5 km/hour on a treadmill.

The results of the resting expiration analysis are shown in Table 7, and the results of the walking expiration analysis are shown in Table 8.

TABLE 7
Resting Expiration Analysis

| | Control group | Catechin | |
|---|---|---|---|
| | n = 7 | group n = 7 | P |
| Energy expenditure | 1936 ± 271 | 2026 ± 274 | 0.5473 |
| (kcal/day) | | | |
| Carbohydrate oxidation | 612 ± 276 | 334 ± 139 | 0.0350 |
| (kcal/day) | | | |
| Fat oxidation (kcal/day) | 997 ± 240 | 1365 ± 273 | 0.0201 |
| Respiratory quotient | 0.81 ± 0.04 | 0.77 ± 0.02 | 0.0174 |

¹Mean ± SD
²For differences across treatments (Student's t-test)

Concerning the resting carbohydrate oxidation, a significant low value was indicated in the catechin group compared with the control group. The resting fat oxidation of the catechin group was about 1.4 times as much as that of the control group, and a significant difference was recognized between the groups. As to the resting respiratory quotient, a significant low value was indicated in the catechin group compared with the control group (Table 7).

TABLE 8
Walking Expiration Analysis

| | Control group | Catechin | |
|---|---|---|---|
| | n = 7 | group n = 7 | P |
| Energy expenditure | 7301 ± 1030 | 8103 ± 849 | 0.1072 |
| (kcal/day) | | | |
| Carbohydrate oxidation | 3012 ± 1062 | 2553 ± 1158 | 0.3424 |
| (kcal/day) | | | |
| Fat oxidation (kcal/day) | 3956 ± 1305 | 5217 ± 904 | 0.0288 |
| Respiratory quotient | 0.83 ± 0.04 | 0.80 ± 0.03 | 0.1187 |

¹Mean ± SD
²For differences across treatments (Student's t-test)

During the walking exercise, the exercising fat oxidation of the catechin group indicated a significantly high value compared with the control group (Table 8).

From the above results, it has been substantiated that the combined use of tea catechin ingestion with regular walking exercise can enhance the oxidation of fat both at resting and during exercise.

1. A method for enhancing physical activities, comprising administering an effective amount of catechins.

2. A method for promoting oxidation of fat in a subject in need thereof, comprising enhancing effects of exercise by a combined use of an administration of an effective amount of catechins and regular exercise.

3. A method for inhibiting obesity in a subject in need thereof, comprising enhancing effects of exercise by a combined use of an administration of an effective amount of catechins and regular exercise.

4. A method for inhibiting liver enlargement in a subject in need thereof, comprising enhancing effects of exercise by a combined use of an administration of an effective amount of catechins and regular exercise.

5. A method for inhibiting diabetes in a subject in need thereof, comprising enhancing effects of exercise by making combined use of an administration of an effective amount of catechins and regular exercise.

6. A method for preventing or ameliorating obesity and/or diabetes in a subject in need thereof, comprising effectively exhibiting anti-obesity effect and/or anti-diabetes effect by a combined use of an administration of an effective amount of catechins and regular exercise.

7. The method of claim 1, wherein said catechins is one or more catechins selected from the group consisting of gallocatechin, catechingallate, gallocatechingallate, epicatechin, epigallocatechin, epicatechingallate and epigallocatechingallate.

8. The method of claim 1, wherein said catechins are in the form of a water or hot water extract of green tea leaves.

9. The method of claim 1, wherein said effective amount ranges from 100 to 3,000 mg/60 kg body-weight of said subject in need thereof, per day.

10. The method of claim 2, wherein said catechins is one or more catechins selected from the group consisting of gallocatechin, catechingallate, gallocatechingallate, epicatechin, epigallocatechin, epicatechingallate and epigallocatechingallate.

11. The method of claim 2, wherein said catechins are in the form of a water or hot water extract of green tea leaves.

12. The method of claim 2, wherein said effective amount ranges from 100 to 3,000 mg/60 kg body-weight of said subject in need thereof, per day.

13. The method of claim 3, wherein said catechins is one or more catechins selected from the group consisting of gallocatechin, catechingallate, gallocatechingallate, epicatechin, epigallocatechin, epicatechingallate and epigallocatechingallate.

14. The method of claim 3, wherein said catechins are in the form of a water or hot water extract of green tea leaves.

15. The method of claim 3, wherein said effective amount ranges from 100 to 3,000 mg/60 kg body-weight of said subject in need thereof, per day.

16. The method of claim 4, wherein said catechins is one or more catechins selected from the group consisting of gallocatechin, catechingallate, gallocatechingallate, epicatechin, epigallocatechin, epicatechingallate and epigallocatechingallate.

17. The method of claim 4, wherein said catechins are in the form of a water or hot water extract of green tea leaves.

18. The method of claim 4, wherein said effective amount ranges from 100 to 3,000 mg/60 kg body-weight of said subject in need thereof, per day.

19. The method of claim 5, wherein said catechins is one or more catechins selected from the group consisting of gallocatechin, catechingallate, gallocatechingallate, epicatechin, epigallocatechin, epicatechingallate and epigallocatechingallate.

20. The method of claim 5, wherein said catechins are in the form of a water or hot water extract of green tea leaves.

21. The method of claim 5, wherein said effective amount ranges from 100 to 3,000 mg/60 kg body-weight of said subject in need thereof, per day.

22. The method of claim 6, wherein said catechins is one or more catechins selected from the group consisting of gallocatechin, catechingallate, gallocatechingallate, epicatechin, epigallocatechin, epicatechingallate and epigallocatechingallate.

23. The method of claim 6, wherein said catechins are in the form of a water or hot water extract of green tea leaves.

24. The method of claim 6, wherein said effective amount ranges from 100 to 3,000 mg/60 kg body-weight of said subject in need thereof, per-day.
